(19) United States
(12) Patent Application Publication
(10) Pub. No.: US 2011/0081641 A1
(43) Pub. Date: Apr. 7, 2011

(54) Method and Apparatus for Automating Chemical and Biological Assays

(76) Inventor: Jason Gould, Mullica, NJ (US)

(21) Appl. No.: 12/573,418

(22) Filed: Oct. 5, 2009

(51) Int. Cl. C12Q 1/00; G01N 33/53; G01N 31/20; G01N 31/22; G01N 21/75; C12M 1/34

(52) U.S. Cl. 435/4; 436/164; 435/287.1; 422/403; 422/423

(57) ABSTRACT

A device which collects specimen fluids or performs chemical or biological assays of the specimen fluid is provided with a specimen fluid receiver and a fluid actuated trigger coupled to receive specimen fluid from the specimen fluid receiver. The trigger is made of a material which expands substantially upon absorbing specimen fluid, and it is mounted and positioned so as to contact and move another component of the device upon expanding through the absorption of specimen fluid. Preferably, the trigger is mounted to the other component and is positioned to press against a stationary surface upon expanding, so that the trigger causes the other component to move. Also, the other component may contain a surface coupled to receive specimen fluid from the specimen fluid receiver and the surface has an area which contains a substance which interacts with the specimen fluid. The trigger is coupled to receive specimen fluid from the specimen fluid receiver in such a manner that there is a predetermined delay before the trigger expands sufficiently to move the other component, and the specimen fluid interacts with the substance during delay.

### BACKGROUND OF THE INVENTION

The present invention relates generally to the performance of chemical and biological assays and, more particularly, concerns a method and apparatus which permit the performance of complex, multistep assay procedures automatically, in a single operator-initiated process.

Monitoring and managing the public health depends very much upon the ability to perform chemical and biological assays, for example immunological assays, reliably and efficiently. In some instances, a health worker must obtain patient specimens, for example, saliva or urine samples, in the field, usually with a handheld collection device. Once the sample is taken, it must be retained safely and securely until it can be delivered to a central location. Often, it is desirable to add a substance to a specimen close to the time that it is taken. Most often, with devices that are to be inserted in the patient's mouth, such substances are added manually by an operator after the sample is taken, owing to the danger that substances which may be harmful may be communicated back to the patient (his mouth) through the collection device. On the other hand, it would be desirable for that substance to be contained in the collection device, both for convenience and to avoid any damage that may result from operator error.

Thus, there is a need for a collection device that can be isolated from the patient when a sample is taken, both for the security of the sample and to prevent communication back to the patient of substances contained in the collection device. Moreover, it is important that such isolation occur automatically in order to prevent accidental damage to specimens or accidental injury to patients.

Chemical and biological assay devices and processes are known which accomplish complicated multistep processes in a single procedure. One example of such assays is “lateral flow” assays. However, it is often necessary or desirable to introduce a delay (an “incubation period”) after one step is performed and before the next one begins. Similarly, additives, for example, running buffer, may need to be introduced into a process after a certain delay. The operator must, for example, take a sample, add an additive, wait a prescribed amount of time, and then perform some other step. This demands diligence and skill on the part of the operator, not to mention rigorous training, as any inattention or error on his part can compromise the entire process. That is, waiting too long, or not long enough, can result in compromising the test results.

It would be desirable to have a multistep process involving delays between steps proceed automatically once it is initiated by an operator. This would not only improve the reliability and consistency of results, but it would allow the process to be performed by an operator with a relatively low level of skill and training in medical technology, for example a police officer or a fireman. It would be particularly desirable to have a handheld device into which a specimen could be introduced, after which the entire process would proceed automatically.

### SUMMARY OF THE INVENTION

The foregoing and other advantages are achieved in accordance with the present invention which relates to a testing device that has a time trigger. The trigger is preferably made of a material which expands substantially upon absorbing specimen fluid, and it is mounted and positioned so as to contact and move another component of the device upon expanding through the absorption of specimen fluid.

Preferably, the trigger is mounted to the other component and is positioned to press against a stationary surface of the device upon expanding, so that the trigger causes the other component to move.

Preferably, the other component contains a surface coupled to receive specimen fluid from the specimen fluid receiver and has an area which contains a substance which interacts with the specimen fluid. The trigger is coupled to receive specimen fluid from the specimen fluid receiver in such a manner that there is a predetermined delay before the trigger expands sufficiently to move the other component, the specimen fluid interacting with the substance during the delay.

Preferably, a second component of the device is positioned to be contacted by the other component is constructed to absorb from the other component specimen fluid which has interacted with the substance. The second component may include an area containing a second substance, where interaction of specimen fluid with the second substance occurs automatically subsequent to the delay.

### BRIEF DESCRIPTION OF THE DRAWINGS

The foregoing brief description and further objects, features and advantages of the present invention will be understood more completely from the following detailed description of presently preferred, but nonetheless illustrative, embodiments in accordance with the present invention, with reference being had to the accompanying drawings in which:

FIGS. 1A and 1B are perspective views of a fluid actuated trigger embodying the present invention, with FIG. 1A showing the trigger in an non-expanded state and FIG. 1B showing the trigger in its fully expanded state, after having been saturated with a liquid, or moist vapor;

FIG. 2 is a perspective view of a first embodiment 20 of a device for performing biological assays in accordance with the present invention;

FIG. 3 is a partially cut-away perspective view of the device 20 of FIG. 2 showing internal construction;

FIGS. 4A and 4B are schematic representations of the internal construction of the device 20 of FIG. 3, useful in describing the operation of the device, with FIG. 4A showing device 20 prior to the absorption of sample liquid by an internal trigger and FIG. 4B showing the device after absorption of the liquid;

FIG. 5 is a partially cut away perspective view of a second embodiment 120 of an assay device in accordance with the present invention;

FIGS. 6A, 6B and 6C are schematic representations of the internal construction of the device 120 of FIG. 5, useful in describing the operation thereof, with FIG. 6A showing device 120 prior to the absorption of sample liquid by an internal trigger, FIG. 6B showing the device after absorption of the liquid, and FIG. 6C showing the device a predetermined time after the absorption of liquid has started;

FIGS. 7A and 7B are schematic representations of the internal construction of an alternate embodiment 20′ of test device 20, useful in describing the operation of device 20′, with FIG. 7A showing device 20′ prior to the absorption of sample liquid by an internal trigger and FIG. 7B showing the device after absorption of the liquid;

FIGS. 8A and 8B are schematic representations of another embodiment 220 of an assay device in accordance with the present invention, with FIG. 8A showing device 220 prior to the absorption of sample liquid by an internal trigger and FIG. 8B showing the device after absorption of the liquid;

FIG. 9A is a partial perspective view showing the forward portion of an assay device which is a secure sample collector 50 embodying the present invention;

FIGS. 9B and 9C are schematic representations of the internal construction of collector 50 of FIG. 9A, with FIGS. 9A and 9B showing collector 50 prior to and subsequent to the absorption of liquid by an internal trigger;

FIG. 10A is a partial perspective view of the forward portion of an ultimate embodiment 150 of a secure collector in accordance with the present invention; and

FIGS. 10B and 10C are schematic representations of the internal construction of collector 150 useful in describing its operation.

### DETAILED DESCRIPTION OF THE PRESENTLY PREFERRED EMBODIMENTS

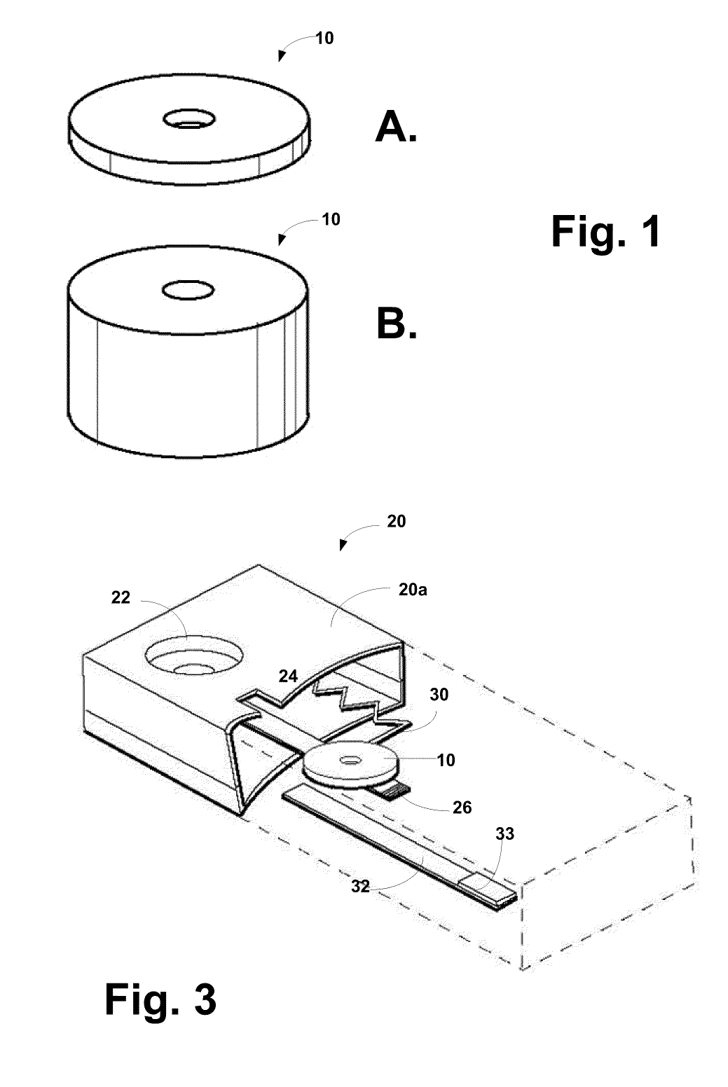

Turning now to the drawings, FIG. 1A is a perspective view of a fluid actuated trigger 10 embodying the present invention. Trigger 10 is preferably in the form of a disc made of compressed cellulose, or some other material that expands substantially in volume when it absorbs or is saturated with a liquid, usually aqueous in nature. Some liquids, such as alcohol, may not operate to expand cellulose material, but any material that can be expanded by any liquid may be used. While FIG. 1A illustrates trigger 10 in its initial state, FIG. 1B illustrates the trigger in its expanded state, after having absorbed a liquid, or the like.

One compressed disk 0.1 to 0.2 mm expands to 1.5 mm. Multiple disks add force and length. Force is uni-dimensional. A preferred material for use in practice of the present invention is the compressed cellulose material is manufactured by Blue Green Ind., Corp. with the following specifications:

Cellulose Sponge, Compressed,

100% Hydrocellulose (regenerated cellulose)

No additives

Color: White

Tear Strength: 8-10 lb (1×¼ inch section wet)

Pore size: 30-50 Durometer (Shore A Compressed Dry)

Elongation: 2% (Wet)

Compression Set: 10 to 1 (Dry)

Heat Resistance: 280 degrees F. continuous

Water Absorption: 15-17 times by weight (from dry state)

Density: 1.3-2.4 lb/ft3

Visual: Middle hole should be centered

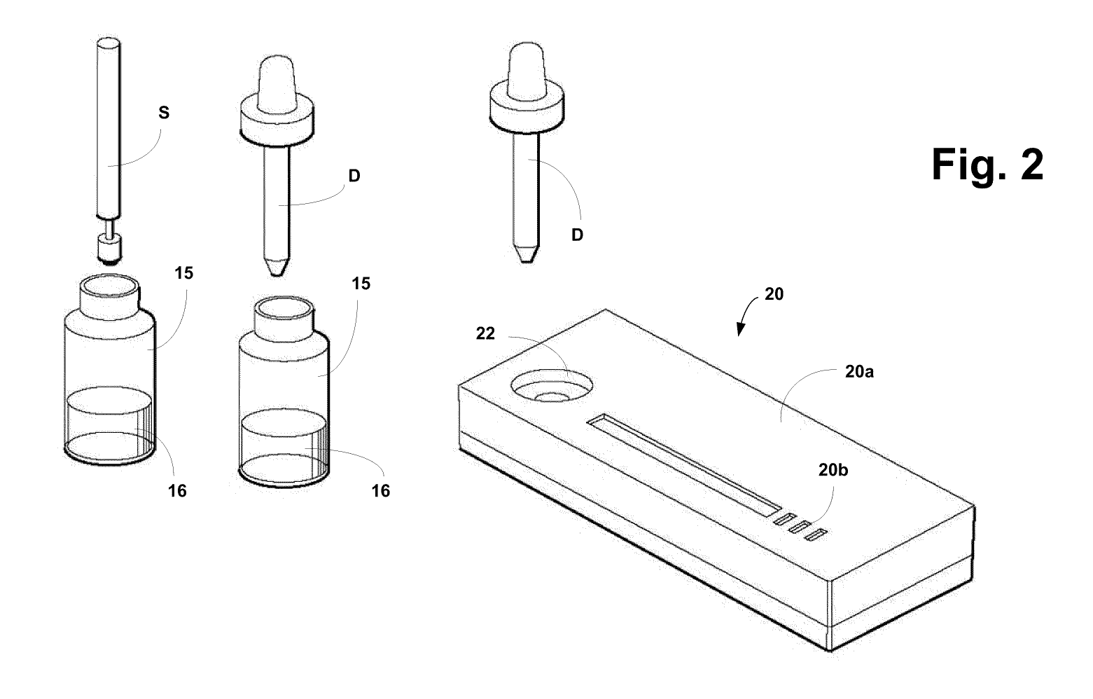

In accordance with one aspect of the present invention, a fluid actuated trigger is utilized to impart movement to components of an assay device. For example, FIG. 2 is a perspective view of a preferred device 20 for performing a biological assay. Initially, a biological specimen is taken with a sampler S and introduced into a container 15 containing a running buffer 16. Using a dropper D, or the like, the buffered specimen is introduced to an inlet well 22 of device 20.

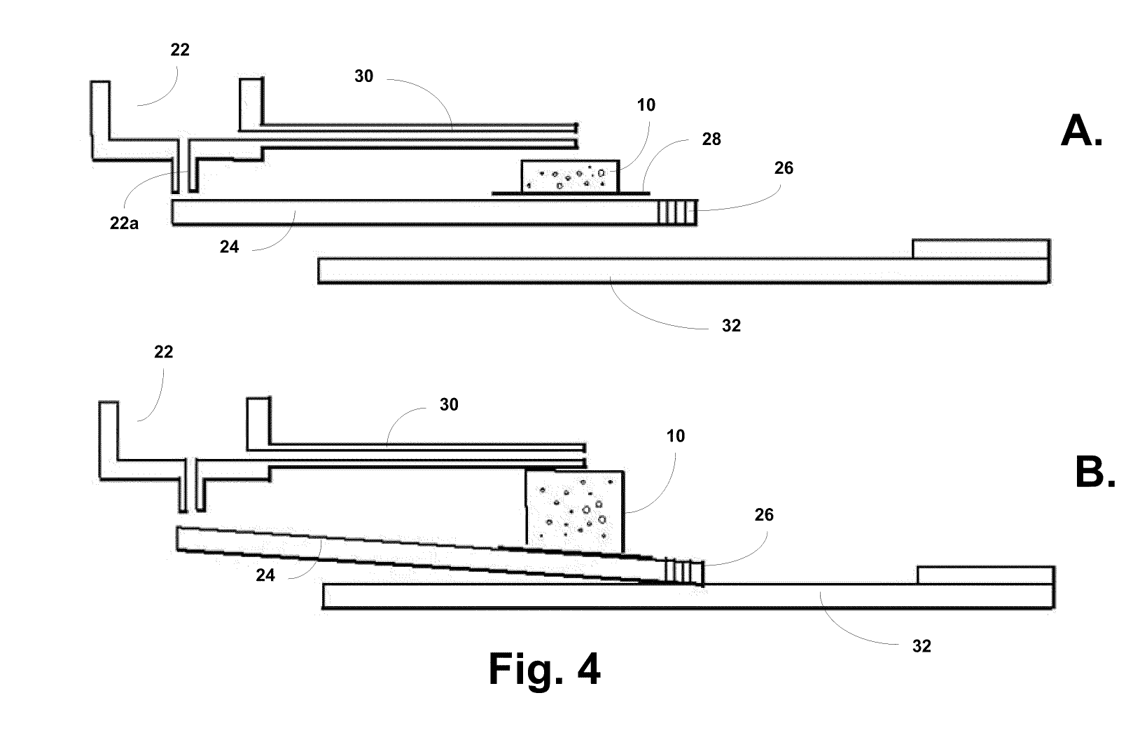

FIG. 3 is a partially cut away perspective view of the device 20 showing internal construction, and FIGS. 4A and 4B are schematic representations of that construction useful in describing the operation of device 20. The solution within the well 22 is dispensed via a capillary outlet 22a onto a sample pad 24 containing a treatment material, for example, a gold conjugate 26. Solution on pad 24 eventually reaches gold 26 and begins to incubate with the gold, in time producing an incubated liquid. A fluid actuated trigger 10 is mounted on pad 24, with a barrier 28 interposed between them that is impermeable to liquid from pad 24. Preferably, barrier 28 is a section of double-sided tape, also utilized to retain trigger 10 in position.

A capillary tube 30 is connected between the well 22 and trigger 10, allowing liquid from well 22 to be introduced gradually to trigger 10. As trigger 10 absorbs liquid from well 22, it begins to swell, bearing upon the stationary undersurface of the top wall 20a of device 20 and forcing pad 24 to bend downward, as illustrated in FIG. 4B. A test strip 32 preferably made of nitrocellulose is mounted at a fixed position below pad 24 and eventually pad 24 bends sufficiently to come into contact with test strip 32, distributing the incubated solution to it. Typically, test strip 32 would be treated with a reagent 33 intended to react with the incubated solution on pad 24. The reagent may for example change color to indicate the results of a test. The treated area 33 may be observed through a window 20B in device 20, as shown in FIG. 2. By design, the dimensions of capillary tube 30 and the saturation time of trigger 10 are calculated to permit complete incubation on pad 24 before it comes into contact with strip 32.

Those skilled in the art will appreciate that, through the use of trigger 10 as disclosed, it becomes possible to perform automatically a two step operation with a programmed delay between the steps. This eliminates the inconsistency and errors that can be introduced when those steps are performed manually by an operator. It also makes it possible for the entire test to be performed successfully by a relatively unskilled operator.

The proper width, size and shape of the various channels within the apparatus can be determined via empirical measurements. Thus, if the expansion occurs to quickly to allow for the proper reaction time, for example, one can simply diminish the size of the channel that provides liquid to the mater for expansion.

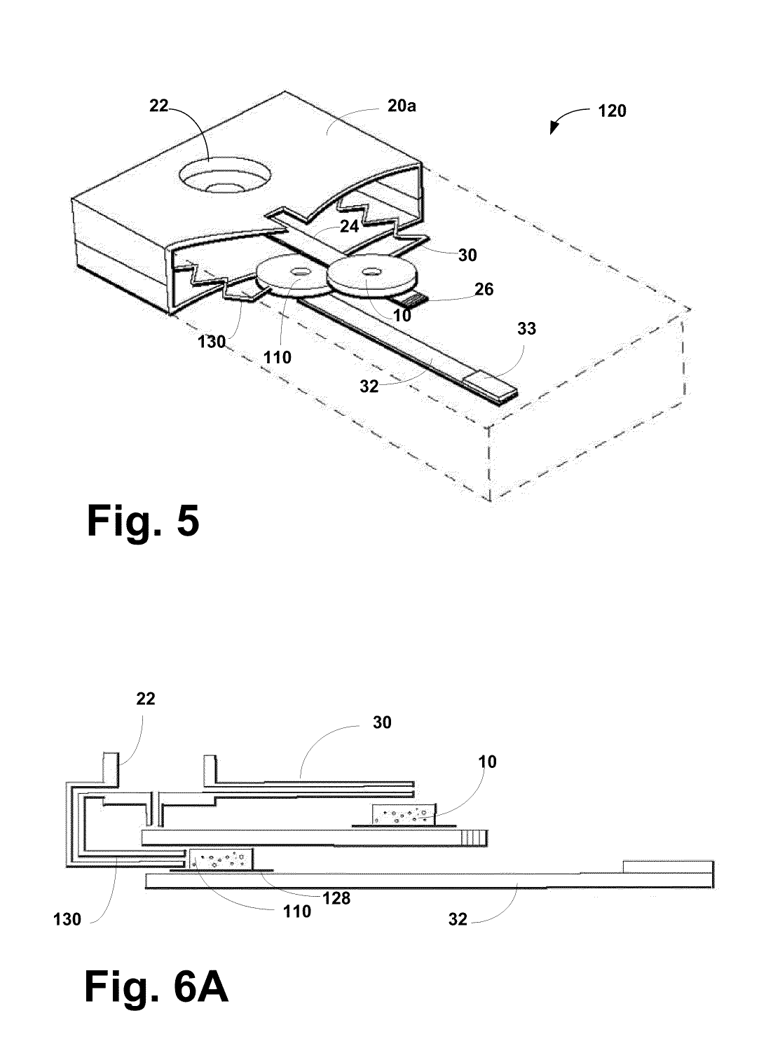

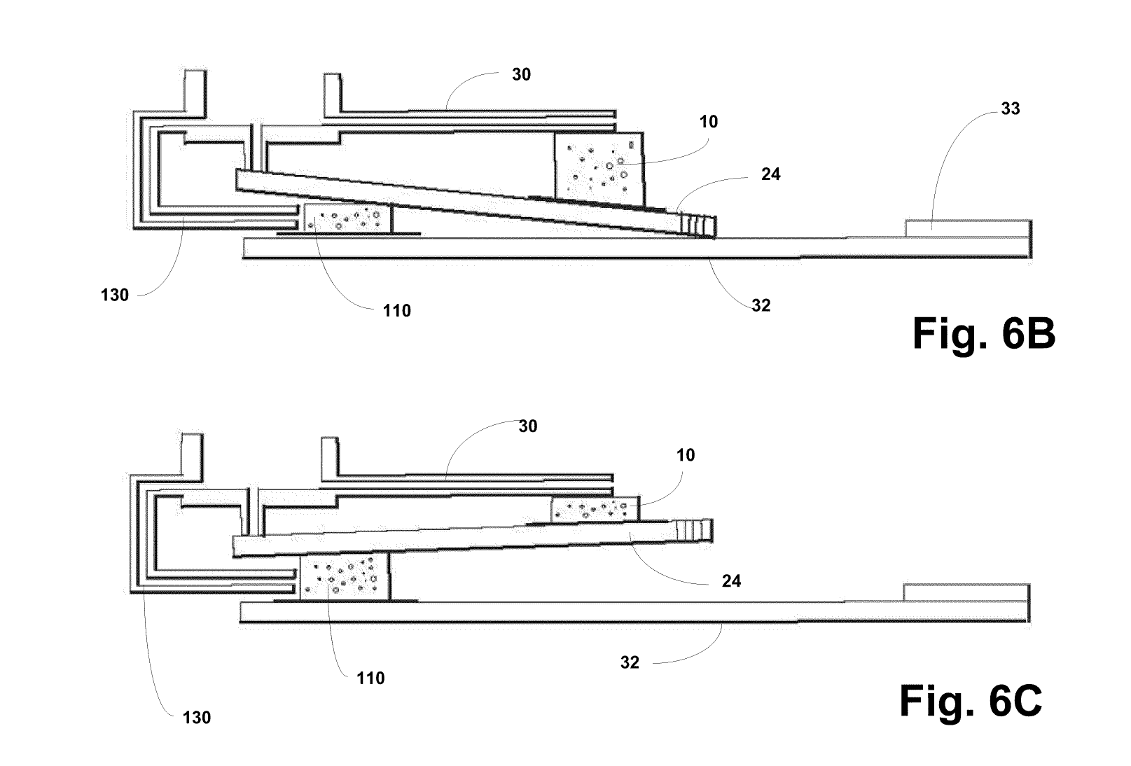

It should be appreciated that, by adding additional liquid actuated triggers, it would be possible to have additional steps performed in a testing device, all with their own timing. For example, FIG. 5 is a partially cut away perspective view of a second embodiment 120 of a testing device in accordance with the present invention. In part, device 120 is identical to device 20, and the identical elements are represented by the same reference characters as in device 20. The primary difference is that device 120 includes a second liquid actuated trigger 110, which is connected to well 22 through a capillary tube 130. FIGS. 6A, 6B and 6C are schematic representations of the internal construction of device 120, useful in describing the operation thereof.

To the extent illustrated in FIGS. 6A and 6B, the operation of device 120 whereby a test indication is provided in treated area 33 is identical to that of device 20. The description already provided with respect to FIGS. 4A and 4B is equally applicable and will not be repeated here. The second liquid actuated trigger 110 is mounted on test strip 32 by means of a second double-sided tape, or the like (ex. friction pins), 128 which holds it in position and also acts as an impermeable barrier. A sample introduced into well 22 will be introduced to trigger 110 through tube 130. As a result, trigger 110 will begin to swell. At a time determined by the construction of tube 130, trigger 110 will have swelled enough to cause separation of pad 24 and strip 32, at which point incubated solution is no longer provided to strip 32. By design, tube 130 will be constructed so that trigger 110 will not act for a sufficient time to permit strip 32 to perform its test. However, tube 130 will also be of such a construction as to assure that pad 24 and strip 32 will be separated after a predetermined time. This will assure that too much incubated solution is not provided to strip 32. For some reactions, providing too much incubated fluid could cause inaccuracies or be detrimental to the reaction taking place on strip 32. Thus, test device 120 assures that there is sufficient incubation on pad 24 before it comes into contact with strip 32, that contact between pad 24 and strip 32 is for a sufficient time to provide an adequate amount of incubated solution, and that the contact is not for such a long time as to provide too much incubated solution. At the same time, the operation of test device is entirely automatic once well 22 is filled and does not require skill or diligence on the part of the operator.

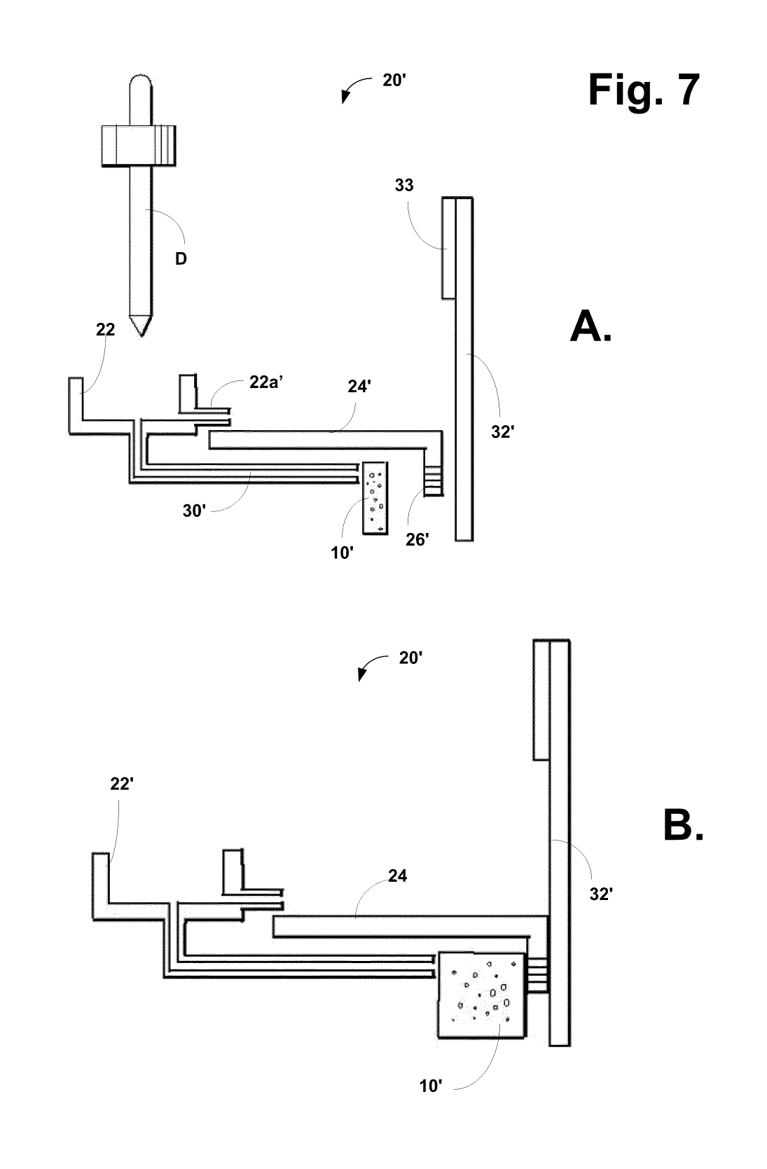

FIGS. 7A and 7B are schematic representations of an alternate embodiment 20′ of test device 20. In this embodiment, well 22 is coupled to a liquid actuated trigger 10′ through a capillary tube 30′. A pad 24′ with gold 26′ thereon is mounted for lateral movement, either with or against gravity, and a test strip 32′ is positioned vertically at a lateral distance from pad 24′. A solution to be tested is provided to well 22, for example with a dropper D, and is deposited upon pad 24′ through an outlet 22a′. Solution applied to pad 24′ will cooperate with gold 26′ to produce an incubated solution. Fluid supplied through tube 30′ causes trigger 10′ to swell and, in time, it will contact on pad 24′, forcing it to the right, into contact with strip 32′. This will cause incubated solution to be applied to strip 32′, and a predetermined test will be performed on the strip, with treated portion 33 ultimately showing the intended test result. As was the case with tube 30, tube 30′ is designed to assure a sufficient incubation time on pad 24′ before pad 24 touches strip 32′.

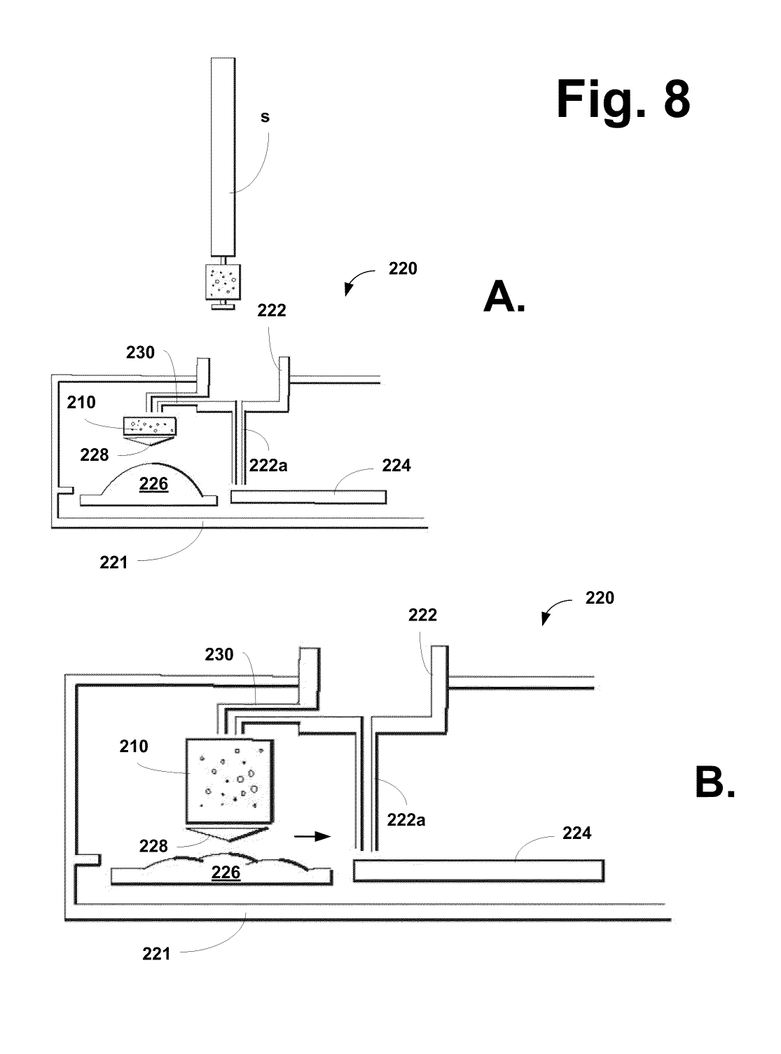

FIGS. 8A and 8B are schematic representations of another embodiment 220 of an assay device in accordance with the present invention. Device 220 includes a hollow body 221 and a well 222. A specimen liquid to be tested may be introduced to well 222, for example with a sample S. Within body 221, there is provided a test strip 224 which, will typically include an indicating portion (not shown) reflecting the result of the assay. Also within body 221, there is provided a package 226 containing a reagent to be applied to strip 224. Positioned above package 226 is a liquid actuated trigger 210, to the bottom of which is attached at element 228, for example a piercing element, to open package 226. A solution introduced to well 222 is introduced onto test strip 224 through outlet 222a. At the same time, liquid is also introduced to trigger 210 through a capillary tube 230 and begins swelling trigger 210. At the same time, test strip 224 is adequately loaded with a specimen liquid. At a time determined by the construction of capillary tube 230, element 228 is forced into package 226, breaking it open and allowing the reagent therein to leak upon test strip 224 as indicated by the arrow. This reagent is then absorbed by the test strip, allowing the intended test to take place.

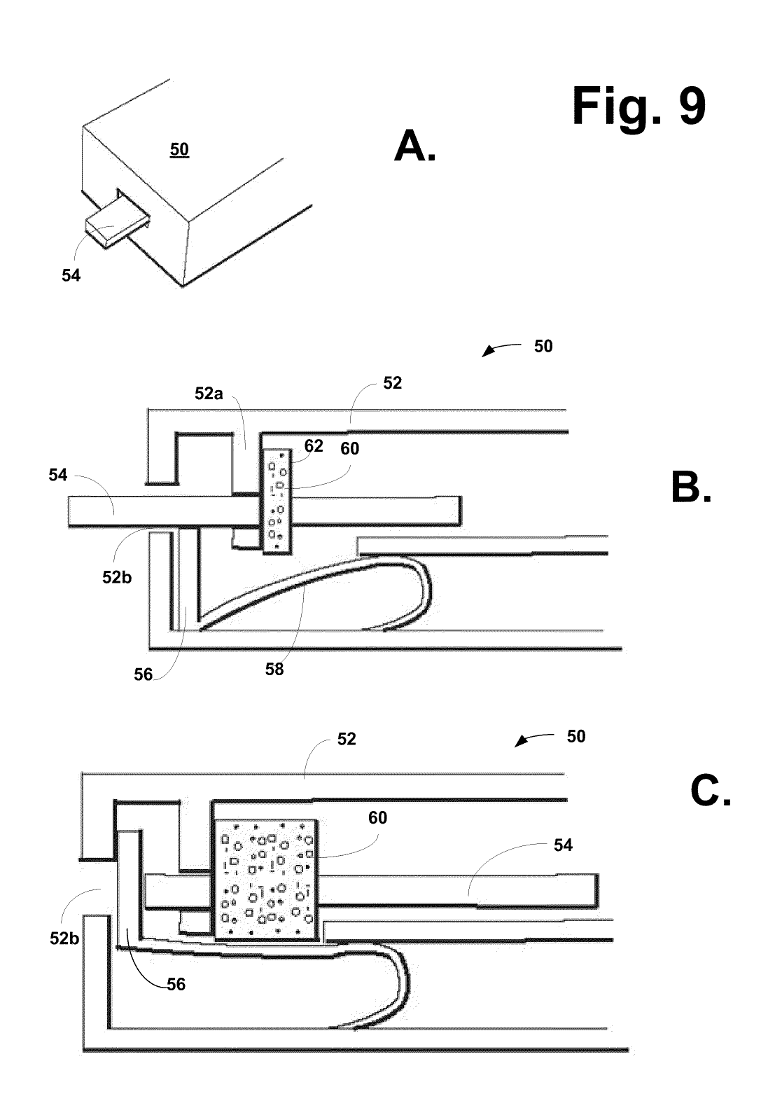

In addition to providing an automatic fluid testing device, a liquid trigger can provide a secure specimen collecting device. For example, FIG. 9A is a partial perspective view showing the forward portion of a secure sample collector 50 embodying the present invention. Collector 50 includes an enclosure or body 52 from which a sample pad 54 protrudes. Collector 50 may be used to collect saliva samples by placing pad 54 on the tongue and saturating it with saliva. Collector 50 is a secure collector, in that, once pad 54 is saturated, it will be withdrawn into the enclosure 52, protecting it against damage and contamination.

FIGS. 9B and 9C are schematic representations of the internal construction of collector 50. Strip 54 protrudes forwardly out of the enclosure 52 through a window 52b. In addition, enclosure 52 contains an internal upright stationary wall 52a and pad 54 protrudes through an opening in that wall and moves freely therein. To the rear of wall 52a, a liquid actuated trigger 60 is mounted on pad 54 so that its rear portion 52 is secured to the pad. Forward of portion 62, however, trigger 60 may move freely over pad 54. An upright door 56 is mounted within enclosure 52 by means of a resilient loop 58 which urges it upward. However, with pad 54 in its pre-use position, door 56 is retained in a downward position (FIG. 9B) below pad 54.

When pad 54 is placed in a patient's mouth to take a saliva sample, the pad begins to absorb liquid, and that liquid is transferred to trigger 60. Trigger 60 begins to expand, with its forward face bearing on wall 52a and since the rear portion 62 is secured to pad 54, pad 54 is drawn rearward into an enclosure 52 through the expansion of trigger 60 (FIG. 9C). When the front of pad 54 passes rearward of door 56, the resilience of loop 58 forces door 56 upward, closing off the window 52b and protecting pad 54 in a sealed compartment.

It will be appreciated that device 50 is not only a secure collecting device, but it would also make it possible to perform tests inside it, without the risk that internal reagents might find there way onto pad 54 and into the patient's mouth. For example, the rear portion of device 50 could include structure such as shown in FIG. 8A to apply a reagent to pad 54 after door 56 is closed.

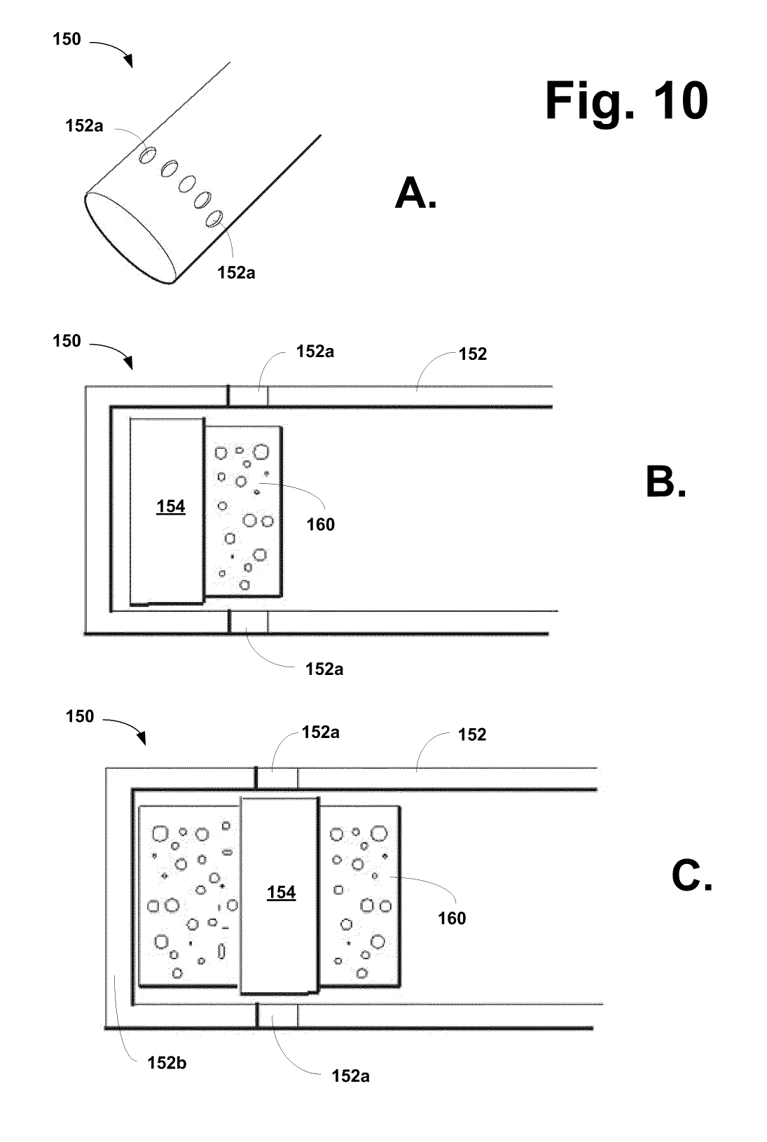

FIG. 10A is a partial perspective view of the forward portion of an alternate embodiment 150 of a secure collector in accordance with the present invention. FIGS. 10B and 10C are schematic representations of the internal construction of collector 150 useful in describing its operation. Collector 150 has a generally cylindrical enclosure 152 containing an array of sampling ports 152a providing access to the interior of the enclosure 152. A generally cylindrical sleeve 154 is mounted within enclosure 152 for longitudinal sliding movement. Mounted inside sleeve 154 is a compressed cellulose plug (trigger) 160 which is secured to the rear of sleeve 154. The forward portion of cellulose plug 160 extends freely into the interior sleeve 154.

In operation, a saliva sample may be taken by placing the forward end of collector 150 into the mouth and saturating it with the tongue. Saliva then seeps through the ports 152a, into the cellulose plug 160. As plug 160 absorbs liquid, it begins to expand, and its forward portion bears against the forward wall 152b of enclosure 152, forcing sleeve 154 rearward. Eventually, sleeve 154 reaches the position shown in FIG. 10C, where it blocks the ports 152a, and no further liquid can be absorbed. In addition, collected saliva remains in the cellulose plug 160, protected by the enclosure 152. As was the case with device 50, the right hand portion of device 150 could include structure such as that shown in FIG. 8A to apply a reagent to plug 160 after sleeve 154 clocks ports 152a.

While the above describes the preferred embodiment of the invention, various other modifications and additions will be apparent to those of skill in the art. Such variations are intended to be covered by the following claims.

1. An assay device for specimen fluid, comprising:
a specimen fluid receiver constructed to receive and retain specimen fluid; another component; and
a fluid actuated trigger coupled to receive specimen fluid from the specimen fluid receiver, said trigger being made of a material which expands substantially upon absorbing specimen fluid, said trigger being mounted and positioned so as to co-act with said another component upon expanding through the absorption of specimen fluid.

2. The assay device of claim 1 wherein said another component is mounted within the device so as to be movable, said trigger is mounted to said another component and is positioned to press against a stationary surface of the device upon expanding, whereby said trigger causes said another component to move.

3. The assay device of claim 1 wherein said another component contains a surface coupled to receive specimen fluid from said specimen fluid receiver and has an area which contains a substance which interacts with said specimen fluid, said trigger being coupled to receive specimen fluid from said specimen fluid receiver in such a manner that there is a predetermined delay before said trigger expands sufficiently to contact and move said another component, said specimen fluid interacting in with said substance during the delay.

4. The assay device of claim 3 further comprising a second component of the device positioned to be contacted by said another component after it moves, said second component being constructed to absorb from said another component specimen fluid which has interacted with said substance.

5. The assay device of claim 4 further comprising an active area on said second component, said active area containing a second substance which can interact with said specimen fluid, whereby interaction of specimen fluid from said another component with said second substance occurs automatically subsequent to said delay.

6. The assay device of claim 5 further comprising a second fluid actuated trigger coupled to receive specimen fluid from said specimen fluid receiver, said second trigger being made of a material which expands substantially upon absorbing specimen fluid and being mounted at a distance from said second component but close enough to contact it upon expansion, said second trigger being coupled to receive specimen fluid from said specimen fluid receiver in such a manner that there is a predetermined second delay before said second trigger expands sufficiently to contact said second component, causing said second component to move after said second delay.

7. The assay device of claim 5 further comprising a second fluid actuated trigger coupled to receive specimen fluid from said specimen fluid receiver, said second trigger being made of a material which expands substantially upon absorbing specimen fluid and being mounted so as to be interposed between said another component and said second component, said second trigger being coupled to receive specimen fluid from said specimen fluid receiver in such a manner that there is a predetermined second delay before said second trigger expands sufficiently to contact said another component and said second component, said second trigger causing said another component and said second component to separate after said second delay.

8. The assay device of claim 1 further comprising:
an enclosure;
said specimen fluid receiver being mounted for movement within said enclosure from a forward position in which it protrudes forwardly from the device to receive specimen fluid, to a rearward position in which it withdrawn into said enclosure;
said another component being a forward wall of said enclosure;
said trigger being mounted to said specimen fluid receiver to be rearward of the forward wall when said specimen receiver is in said forward position but to be free to expand towards said forward wall, said trigger moving said specimen fluid receiver rearward upon expanding to contact said forwarded wall, whereby said fluid receiver is withdrawn into said enclosure.

9. The assay device of claim 8 further comprising:
a window in said enclosure through which said specimen fluid receiver protrudes; a door mounted within said enclosure for movement crosswise to the movement of said specimen fluid receiver, said door being positioned to expose said window; and
resilient means urging said door to cover said window;
said specimen fluid receiver preventing said door from covering said window when in its forward position, said door covering said window under the action of said resilient means when said specimen fluid receiver is in its rearward position.

10. The assay device of claim 1 further comprising:
an enclosure having an access port to an interior thereof proximate a forward end thereof;
said another component being a cover mounted in said enclosure for movement from a first position in which said port is uncovered to a second position in which it covers said port;
said trigger acting as said specimen fluid receiver by receiving fluid through said port and being mounted to said cover so as to move it from its first to its second position upon expanding through the absorption of specimen fluid.

11. The assay device of claim 10 wherein the port is rearward of said forward end, which is defined by a forward wall of said enclosure, said first position of said cover being forward of said port, said trigger being mounted to said cover and upon expansion contacting said forward wall so as to move said cover rearward.

12. The assay device of claim 11 wherein said cover is a sleeve within which said trigger is mounted so as to be free to expand forwardly, said trigger moving said sleeve toward its second position upon expansion.

13. The assay device of claim 1 further comprising a surface coupled to receive specimen fluid from said specimen fluid receiver, an enclosure containing a treatment fluid to be provided to said surface, said another component being an element constructed to act upon said enclosure to cause release of said treatment fluid, said trigger element being mounted to said another component so that, upon expansion, it will cause said another component to act upon said enclosure to release said treatment fluid, said trigger being coupled to receive specimen fluid from said specimen fluid receiver in such a manner that there is a predetermined delay before said trigger expands sufficiently to cause said another component to act upon said enclosure, whereby said treatment fluid is provided to said surface after said delay.

14. A method for introducing operational delay into an assay device for a specimen fluid comprising the steps of:
providing an actuation element which must be moved to operate the device;
providing a fluid actuated trigger coupled to receive specimen fluid, said trigger being made of a material which expands substantially upon absorbing specimen fluid; and
mounting said trigger so as to move said actuation element to operate the device upon expanding by a predetermined amount through the absorption of specimen fluid.

15. The method of claim 14 further comprising coupling the trigger to receive the specimen fluid through a connection which transfers the fluid at a predetermined controlled rate, whereby a delay time before the device is operated can be closely controlled.

16. The method of claim 14 wherein the actuation element includes a receiving surface which receives specimen fluid and has an area containing a substance which interacts with the specimen fluid, the trigger moving the receiving surface into communication with a testing surface, whereby the specimen fluid interacts with the substance for a delay time before the receiving surface is in communication with the testing surface.

17. The method of claim 16 further comprising providing a second fluid actuated trigger coupled to receive specimen fluid, said second trigger being made of a material which expands substantially upon absorbing specimen fluid, mounting said trigger so as to be interposed between said receiving surface and said testing surface so as to separate them upon expanding by a predetermined amount through the absorption of specimen fluid; and controlling the flow of specimen fluid to said second fluid actuated trigger so that it expands by the predetermined amount after a predetermined turnoff delay time.

18. The method of claim 14 utilized in an assay device which has an enclosure, an absorbent specimen fluid collector protruding from the enclosure, the specimen fluid collector being mounted so as to be retractable into the enclosure, said method further comprising mounting the actuation element to the specimen fluid collector and coupling the trigger to the actuation element so that it retracts the specimen fluid collector into the enclosure as the trigger expands and coupling the trigger to the specimen fluid collector so that it receives fluid therefrom.

19. The method of claim 18 utilized in an assay device in which the specimen fluid collector protrudes from the enclosure through a window, further comprising the step of providing a cover in the enclosure which is resiliently biased to cover said window but blocked by the specimen fluid collector, whereby the window is covered automatically when the specimen fluid collector is withdrawn.

20. The method of claim 18 utilized in an assay device which has an enclosure with a port offering fluid access into the interior of the enclosure, the actuator element being a cover which is movable to cover the port, the trigger acting as a specimen fluid collector, said method further comprising mounting the cover to the trigger so that the port is initially uncover but is moved to cover the port upon expansion of the trigger.

21. The method of claim 14 utilized in an assay device having a surface coupled to receive specimen fluid, and an enclosure containing a treatment fluid to be provided to said surface, the actuation element being an element constructed to act upon the enclosure to cause release of said treatment fluid, said method further comprising mounting the actuation element to the trigger so that it will act upon the enclosure when the trigger expands, and coupling the trigger to receive specimen fluid in such a manner that there is a predetermined delay before the trigger expands sufficiently to release the treatment fluid.
